(19) United States
(12) Patent Application Publication
(10) Pub. No.: US 2026/0191486 A1
(43) Pub. Date: Jul. 9, 2026

(54) MEDICAL INFORMATION DISPLAY APPARATUS, X-RAY DIAGNOSTIC APPARATUS, AND MEDICAL INFORMATION DISPLAY CONTROL METHOD

(71) Applicant: Canon Kabushiki Kaisha, Tokyo (JP)

(72) Inventors: Daisuke TAKAHASHI, Yaita (JP); Haruki IWAI, Otawara (JP); Hisanori KATO, Otawara (JP); Takehito TOMARU, Otawara (JP); Masato AKIMOTO, Sakura (JP); Takehiro FUKUZAKI, Utsunomiya (JP); Takayuki TOMISAKI, Nasushiobara (JP)

(73) Assignee: Canon Kabushiki Kaisha, Tokyo (JP)

(21) Appl. No.: 19/414,471

(22) Filed: Dec. 10, 2025

(30) Foreign Application Priority Data
Jan. 6, 2025 (JP) 2025-001754

(51) Int. Cl. A61B 6/46; A61B 6/00; A61B 90/00

(52) U.S. Cl. CPC A61B 6/462; A61B 6/487; A61B 90/36; A61B 2090/365; A61B 2090/368

(57) ABSTRACT

A medical information display apparatus according to an embodiment includes augmented reality glasses and processing circuitry. The augmented reality glasses include a transmissive display area and are capable of displaying medical information in the display area. The processing circuitry acquires the medical information from a medical image collection apparatus, changes a transparency of a medical information image indicating the medical information in accordance with an operation performed on the medical image collection apparatus, and displays the medical information image with the changed transparency in the display area.

### CROSS-REFERENCE TO RELATED APPLICATIONS

This application is based upon and claims the benefit of priority from Japanese Patent Application No. 2025-001754, filed January 6, 2025, the entire contents of which are incorporated herein by reference.

### FIELD

Embodiments described herein relate generally to a medical information display apparatus, an X-ray diagnostic apparatus, and a medical information display control method.

### BACKGROUND

As a display device that displays medical images such as a fluoroscopic image, an endoscopic image, and patient information, conventionally, there has been a display device such as augmented reality (AR) glasses worn by an operator. The display device worn by the operator is used in a state where the front side of the operator is covered. Thus, when the operator uses a medical device, display images displayed on the display device in various conditions may sometimes fail to be appropriately displayed. For example, in a case where a fluoroscopic image is superimposed on a scene visible through the display device, when the transparency of the fluoroscopic image is too low, the fluoroscopic image displayed on the display device when fluoroscopy is unnecessary may become an obstacle to the user. In addition, when the transparency of the fluoroscopic image is too high during fluoroscopy, the image quality of the fluoroscopic image becomes insufficient, and diagnosis based on the fluoroscopic image may become difficult.

### BRIEF DESCRIPTION OF THE DRAWINGS

FIG. 1 is a diagram illustrating an example of an external appearance of a medical image collection system according to an embodiment;

FIG. 2 is a diagram illustrating an example of a configuration of the medical image collection system according to the embodiment;

FIG. 3 is a flowchart illustrating an example of a procedure for medical information display processing according to the embodiment;

FIG. 4 is a diagram illustrating an example of medical information displayed on augmented reality (AR) glasses before and after a fluoroscopy ON according to the embodiment;

FIG. 5 is a diagram illustrating an example of medical information displayed on the AR glasses before and after an image capturing ON according to the embodiment;

FIG. 6 is a diagram illustrating an example of a fluoroscopic image displayed on the AR glasses before and after a change of a line of sight of a user according to a first modified example of the embodiment;

FIG. 7 is a diagram illustrating an example of medical information displayed on the AR glasses before and after a fluoroscopy OFF according to a second modified example of the embodiment;

FIG. 8 is a diagram illustrating an example of medical information displayed on the augmented reality (AR) glasses before and after a fluoroscopy ON according to a third modified example of the embodiment;

FIG. 9 is a diagram illustrating an example of medical information displayed on the augmented reality (AR) glasses before and after a fluoroscopy ON according to a fourth modified example of the embodiment;

FIG. 10 is a diagram illustrating an example of medical information displayed on the augmented reality (AR) glasses before and after a fluoroscopy ON according to a sixth modified example of the embodiment;

FIG. 11 is a diagram illustrating an example of medical information displayed on the AR glasses before and after input of a moving operation of a couch according to a seventh modified example of the embodiment; and

FIG. 12 is a diagram illustrating an example of a configuration of a medical image collection system according to an application example of the embodiment.

### DETAILED DESCRIPTION

A medical information display apparatus according to an embodiment includes augmented reality glasses and processing circuitry. The augmented reality glasses include a transmissive display area and are capable of displaying medical information in the display area. The processing circuitry acquires the medical information from a medical image collection apparatus, changes a transparency of a medical information image indicating the medical information in accordance with an operation performed on the medical image collection apparatus, and displays the medical information image with the changed transparency in the display area.

Various Embodiments will be described hereinafter with reference to the accompanying drawings.

In the following embodiment, a redundant description will be appropriately omitted on an assumption that components assigned the same reference numeral perform similar operations. Hereinafter, an X-ray diagnostic apparatus that performs an examination and a treatment on a gastrointestinal tract, a urinary organ, orthopedics, interventional radiology (IVR), or the like will be described as an example of an X-ray diagnostic apparatus according to the present application. However, embodiments of the present application are not limited thereto.

Further, a medical information display apparatus according to the present application is a glasses-type display device. The medical information display apparatus is used by a user such as a doctor, a technician, or a nurse wearing the medical information display apparatus. The description will be provided on an assumption that glasses in the medical information display apparatus are augmented reality (AR) glasses (hereinafter referred to as AR glasses). The medical information display apparatus, however, is not limited to the AR glasses as long as augmented reality (AR) can be implemented. For example, the medical information display apparatus may be realized by a display or a monitor that can be worn by the user and can implement AR.

### (Embodiment)

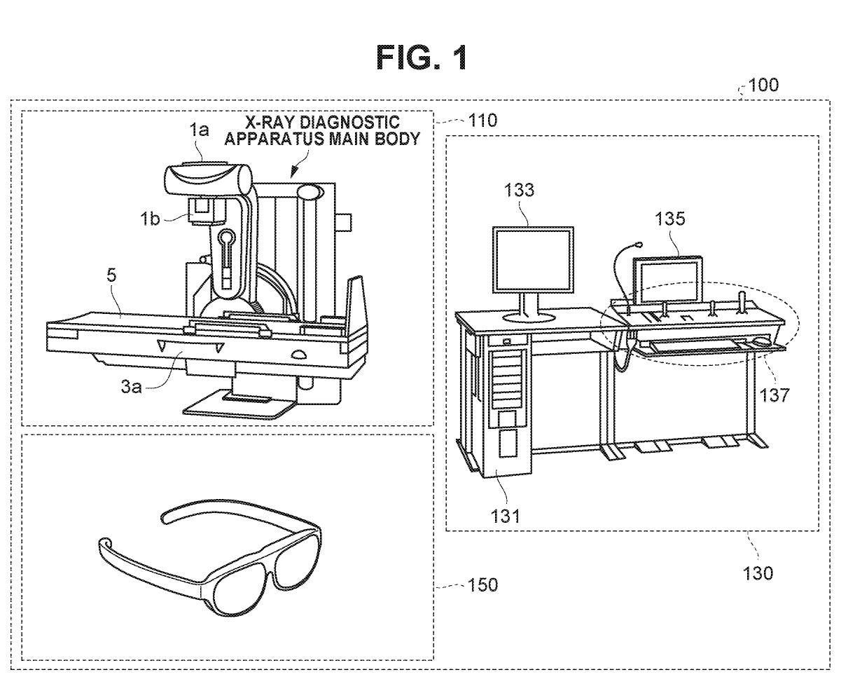

An example of a medical image collection system 100 according to an embodiment will be described with reference to FIG. 1. FIG. 1 is a diagram illustrating an example of an external appearance of the medical image collection system 100 according to the embodiment. As illustrated in FIG. 1, in the medical image collection system 100 according to the embodiment, an X-ray diagnostic apparatus main body (may be simply referred to as an X-ray diagnostic apparatus) 110, a console apparatus 130 including an image processing apparatus 131, a system monitor 133, a fluoroscopic monitor 135, and the like, and a medical information display apparatus 150 are connected to each other in either a wireless or wired manner. The configuration of the medical image collection system 100 is not limited to the configuration illustrated in FIG. 1, and may further include, for example, a proximal operation console including a fluoroscopic monitor or the like.

The X-ray diagnostic apparatus main body 110 includes an X-ray tube 1a that generates X-rays, an X-ray collimator 1b that narrows an X-ray irradiation area, an X-ray detection unit (may also be referred to as an X-ray detector) 3 that detects X-rays, a couch 5 on which a subject P to be irradiated with X-rays is to be placed, and the like.

For example, by operating the console apparatus 130, an operator (user) such as a technician located outside an examination room such as an operation room causes the X-ray diagnostic apparatus main body 110 to perform fluoroscopy and image capturing while concurrently performing operations such as tilting the couch 5 on which a patient (subject) is placed, and vertically moving an imaging system such as the X-ray tube 1a and the X-ray collimator 1b. In this case, the X-ray diagnostic apparatus main body 110 may also be referred to as a fluoroscopy imaging apparatus. In addition, the operator observes a fluoroscopic image displayed on the fluoroscopic monitor 135 included in the console apparatus 130, a captured image and a fluoroscopic image that are displayed on the system monitor 133, and the like. In addition, for example, by operating a proximal operation console installed in an examination room, an operator located in the examination room causes the X-ray diagnostic apparatus main body 110 to perform processing similar to the above-described processing, and observes various images displayed on the fluoroscopic monitor included in the proximal operation console or an examination room monitor.

The console apparatus 130 and the proximal operation console correspond to, for example, a console. As illustrated in FIG. 1, the console (the console apparatus 130 and the proximal operation console) includes an input interface 137 such as various buttons and various levers that are related to control of the X-ray diagnostic apparatus main body 110, and an output device such as various monitors. The output device corresponds to, for example, a display such as the above-described system monitor 133 and the fluoroscopic monitor 135. The input interface 137 will be described below.

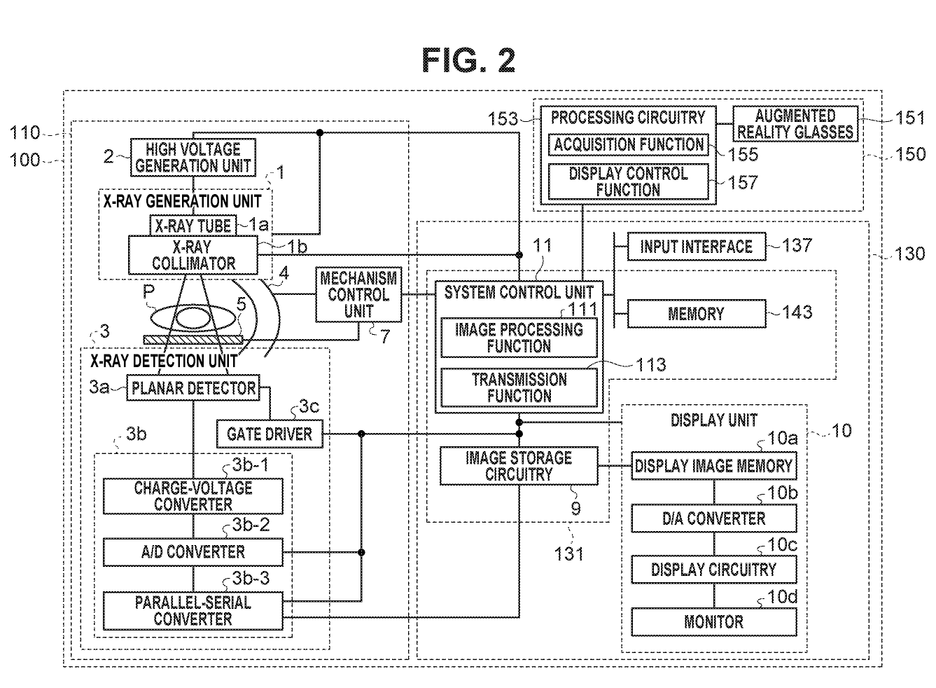

FIG. 2 is a diagram illustrating an example of a configuration of the medical image collection system 100 according to the present embodiment. The medical image collection system 100 includes the X-ray diagnostic apparatus main body 110, the console apparatus 130, and the medical information display apparatus 150. The X-ray diagnostic apparatus main body 110 includes an X-ray generation unit 1, a high voltage generation unit 2, the X-ray detection unit 3, a support arm 4, the couch 5, and a mechanism control unit 7. The X-ray diagnostic apparatus main body 110 and the console apparatus 130 may be collectively referred to as an X-ray diagnostic apparatus.

The X-ray generation unit 1 generates X-rays. The X-ray generation unit 1 includes, for example, the X-ray tube 1a and the X-ray collimator 1b. The X-ray generation unit 1 may also be referred to as an X-ray generation apparatus. The X-ray tube 1a is a vacuum tube that generates X-rays by emitting thermoelectrons from a cathode (filament) toward an anode (target) through application of a high voltage from the high voltage generation unit 2 and supply of a filament current. By thermoelectrons colliding with the target, X-rays are generated. The X-ray tube 1a may be, for example, a rotating anode X-ray tube that generates X-rays by emitting thermoelectrons to a rotating anode. The type of the X-ray tube 1a is not limited to a rotating anode type, and any type of X-ray tube may be applied.

The X-ray collimator 1b shapes an irradiation field of the X-rays from the X-ray tube 1a. The X-ray collimator 1b is provided in front of an X-ray emission window of the X-ray tube 1a. The X-ray collimator 1b includes a plurality of collimator blades. For example, the X-ray collimator 1b includes two collimator blades facing each other, as the plurality of collimator blades. Specifically, the X-ray collimator 1b includes, for example, four collimator blades formed of metal plates of lead or the like. Each of the four collimator blades is driven by a drive device (not illustrated) in accordance with a region of interest input by the operator via the input interface 137.

By sliding the collimator blades using the drive device, the X-ray collimator 1b adjusts the size of a region in which X-rays are shielded to a desired size. By the adjusted collimator blades, the X-ray collimator 1b shields X-rays outside an aperture region. Accordingly, the X-ray collimator 1b narrows the X-rays generated by the X-ray tube 1a so that the X-rays are emitted toward the region of interest of the subject P.

The X-ray collimator 1b includes various X-ray filters such as a beam quality adjustment filter, a compensating filter, and a dose reduction filter. The X-ray filters adjust the dose and the quality of transmitted X-rays for the purpose of reducing an exposure dose of the subject P and improving the image quality of X-ray image data. The X-ray collimator 1b includes a filter movement mechanism that adjusts the positions of the various filters. The filter movement mechanism is implemented by a known mechanism such as various motors and actuators. The filter movement mechanism is driven under the control of a system control unit 11, and adjusts the positions of the various filters.

The beam quality adjustment filter changes the beam quality of transmitted X-rays depending on its material and thickness, thereby reducing soft X-ray components that are easily absorbed by the subject P and reducing high energy components that would otherwise cause a decrease in contrast of the X-ray image data. The compensating filter includes, for example, two filters and attenuates part of the X-rays emitted from the X-ray tube 1a for the purpose of suppressing halation and the like. The dose reduction filter attenuates part of the X-rays emitted from the X-ray tube 1a so that a dose of X-rays to be emitted to a region other than a region of interest becomes a lower dose compared with a dose of X-rays to be emitted to the region of interest, for the purpose of reducing an exposure dose to the subject P and the like.

The high voltage generation unit 2 includes electric circuitry such as a transformer and a rectifier, and an X-ray control device. The high voltage generation unit 2 has a function of generating a high voltage to be applied to the X-ray tube 1a and a filament current to be supplied to the X-ray tube 1a. The high voltage generation unit 2 may also be referred to as a high voltage generation apparatus. The X-ray control device controls an output voltage corresponding to X-rays emitted by the X-ray tube 1a. The high voltage generation unit 2 may adopt either a transformer system or an inverter system. The high voltage generation unit 2 may be provided in the support arm 4.

The X-ray detection unit 3 detects X-rays generated by the X-ray tube 1a. The X-ray detection unit 3 includes, for example, a planar detector 3a corresponding to an X- ray detector, an image data generation unit 3b, and a gate driver 3c. The planar detector 3a is implemented, for example, by a flat panel detector (hereinafter referred to as "FPD"). The FPD includes a plurality of semiconductor detection elements. The semiconductor detection elements include a direct conversion type semiconductor detection element that directly converts X-rays into electrical signals, and an indirect conversion type semiconductor detection element that converts X-rays into light using a phosphor and then converts the light into electrical signals. Either type may be used for the FPD. The electrical signals generated by the FPD are output to the image data generation unit 3b.

The image data generation unit 3b includes a charge-voltage converter 3b-1, an analog to digital (A/D) converter 3b-2, and a parallel-serial converter 3b-3. The charge-voltage converter 3b-1 converts an electrical signal output from the planar detector 3a into a voltage signal, and outputs the voltage signal to the A/D converter 3b-2. Under the control of the system control unit 11, the A/D converter 3b-2 converts an electrical signal, which has been converted into the voltage signal, into X-ray image data as digital data, and outputs the X-ray image data to the parallel-serial converter 3b-3. Under the control of the system control unit 11, the parallel-serial converter 3b-3 converts the A/D-converted X-ray image data from parallel data into serial data, and outputs the serial data to image storage circuitry 9. The image data generation unit 3b may also be referred to as an image data generation apparatus. Here, an X-ray image includes a fluoroscopic image generated by X-ray fluoroscopy of the subject P and a captured image generated by X-ray image capturing of the subject P.

Under the control of the system control unit 11, the gate driver 3c drives detection elements of the planar detector 3a.

The support arm 4 movably supports the X-ray tube 1a that generates X-rays and the planar detector 3a that detects X-rays and is opposite to the X-ray tube 1a. In other words, the X-ray tube 1a and the planar detector 3a are attached to end portions of the support arm 4 so as to face each other. The support arm 4 rotatably supports the X- ray collimator 1b and the planar detector 3a about a rotational axis corresponding to a straight line connecting a focal point where X-rays are generated in the X-ray tube 1a and a central part of the planar detector 3a. By an operation of a support arm movement mechanism, the support arm 4 rotates the X-ray collimator 1b and the planar detector 3a about the rotational axis. The support arm 4 may support the X-ray generation unit 1 and the X-ray detection unit 3 so that, for example, a source-to-image distance (hereinafter referred to as "SID") can be changed.

The couch 5 includes a couchtop and a base. The subject P is placed on the couchtop. The base supports the couchtop so that the couchtop can be translated along, for example, a longitudinal direction of the couchtop, a lateral direction of the couchtop, a vertical direction, and the like. In addition, the base rotatably supports the couchtop about a rotational axis corresponding to the longitudinal direction of the couchtop, the lateral direction of the couchtop, the vertical direction, and the like.

A mechanism unit (not illustrated), which may also be referred to as a mechanism apparatus, includes the support arm movement mechanism and a couch movement mechanism. The support arm movement mechanism and the couch movement mechanism are implemented, for example, by a motor, an actuator, or the like. Under the control of the mechanism control unit 7, the support arm movement mechanism moves the support arm 4 to a position designated by an operator or a preset position. Under the control of the mechanism control unit 7, the couch movement mechanism moves the couchtop of the couch 5 to a position designated by an operator or a preset position. The support arm 4 and the couch 5 correspond to movable parts of the medical image collection apparatus (X-ray diagnostic apparatus main body 110).

Under the control of the system control unit 11, the mechanism control unit 7 controls the mechanism unit. For example, the mechanism control unit 7 controls a moving operation of the support arm movement mechanism and a moving operation of the couch movement mechanism based on an input operation received from the operator via the input interface 137. Specifically, the mechanism control unit 7 reads out a mechanism control program stored in a storage, loads the mechanism control program into a memory in a processor in the mechanism control unit 7, and controls the moving operation of the support arm movement mechanism and the moving operation of the couch movement mechanism in accordance with the loaded mechanism control program. The mechanism control unit 7 may also be referred to as processing circuitry (mechanism control circuitry) that controls the mechanism apparatus.

The image processing apparatus 131 includes the image storage circuitry 9, the system control unit 11, and a memory 143. The image storage circuitry 9 includes a hard disk drive (HDD), a semiconductor memory, and the like. The image storage circuitry 9 stores X-ray image data output from the image data generation unit 3b.

Based on an input operation received from the operator via the input interface 137, the system control unit 11 controls the X-ray generation unit 1, the high voltage generation unit 2, the gate driver 3c, the A/D converter 3b-2, the parallel-serial converter 3b-3, the mechanism control unit 7, the image storage circuitry 9, a display unit 10, and the like. Specifically, the system control unit 11 reads out a system control program, loads the system control program into its memory, and controls each component of the medical image collection system 100 in accordance with the loaded control program. For example, by performing amplification processing, image calculation processing, and the like on the X-ray image data stored in the image storage circuitry 9, an image processing function 111 in the system control unit 11 generates an X-ray image to be displayed.

In accordance with an instruction issued by the operator via the input interface 137, the system control unit 11 sets a region of interest (ROI) in an X-ray image (fluoroscopic image and captured image). In addition, in accordance with an instruction issued by the operator via the input interface 137, the system control unit 11 executes image processing on the entire region or the ROI of the X-ray image by the image processing function 111. For example, a program (image processing program) for executing the image processing is stored in the memory 143. At this time, the image processing function 111 executes the image processing by reading out the image processing program from the memory 143, loading the image processing program into a memory in the system control unit 11, and executing the image processing program. The system control unit (system control circuitry) 11 that implements the image processing function 111 corresponds to an image processing unit. Because known image processing can be used as the image processing, the description thereof will be omitted. Various types of image processing are not limited to being executed by the system control unit 11, but may be implemented by separately-provided image processing circuitry (not illustrated).

In accordance with an image display instruction or the like that has been issued by an operator, a transmission function 113 in the system control unit 11 outputs an X-ray image stored in the image storage circuitry 9 to a display image memory 10a of the display unit 10. In addition, in accordance with an operation performed on the X-ray diagnostic apparatus main body 110, the transmission function 113 transmits the X-ray image to the medical information display apparatus 150. The operation is at least one of an operation related to collection of medical images performed by the X-ray diagnostic apparatus main body 110 and an operation performed by a user wearing augmented reality (AR) glasses 151 of the medical information display apparatus 150.

Hereinafter, for the sake of clarity, the operation is assumed to be an instruction to execute fluoroscopy of the subject P or an instruction to execute image capturing of the subject P, in the operation related to the collection of medical images performed by the X-ray diagnostic apparatus main body 110. For example, in a case where the operation related to the collection of medical images is the instruction to execute fluoroscopy (X-ray fluoroscopy) of the subject P, the transmission function 113 transmits a fluoroscopic image, on which image processing has been performed by the image processing function 111, to the medical information display apparatus 150. In addition, in a case where the operation related to the collection of medical images is the instruction to execute image capturing (X-ray image capturing) of the subject P, the transmission function 113 transmits a captured image (captured X-ray image), on which image processing has been performed by the image processing function 111, to the medical information display apparatus 150.

Other operations related to the collection of medical images, and an operation performed by the user wearing the AR glasses 151 will be described in modified examples. The system control unit 11 is implemented by, for example, processing circuitry (may also be referred to as system control circuitry) that controls each component of the X-ray diagnostic apparatus 110. In addition, the system control unit 11 may also be simply referred to as a control unit.

The display unit 10 includes the display image memory 10a, a digital to analog (D/A) converter 10b, display circuitry 10c, and a monitor 10d. The display image memory 10a stores image data output from the image storage circuitry 9, and outputs the image data to the D/A converter 10b. The D/A converter 10bconverts the image data output from the display image memory 10a into an analog signal, and outputs the analog signal to the display circuitry 10c. The display circuitry 10c displays, on the monitor 10d, an X-ray image based on the image data output from the D/A converter 10b.

The display unit 10 can be implemented as a display and/or various monitors. Specifically, the display unit 10 corresponds to, for example, the system monitor 133, the fluoroscopic monitor 135, and the like in FIG. 1. At this time, for example, a liquid crystal display (LCD), a cathode ray tube (CRT) display, an organic electroluminescence display (OELD), a plasma display, or any other display can be used as the display as appropriate. In addition, the display may be a desktop-type display, or may include a tablet terminal or the like that can wirelessly communicate with the system control unit 11 and the image storage circuitry 9. In addition, in a case where the monitor 10d displays an X-ray image as digital data, the D/A converter 10b becomes unnecessary.

The input interface 137 receives various input operations from the operator, converts the received input operations into electrical signals, and outputs the electrical signals to the system control unit 11. The input interface 137 receives, from the operator, an operation for operating at least one of the support arm 4 and the couch 5, an X-ray condition related to generation of X-rays, a condition related to image processing, an operation of the four collimator blades, and the like. As the input interface 137, for example, a mouse, a keyboard, a trackball, a switch, a button, a joystick, a foot switch, a touch pad, a touch panel display, and the like can be used as appropriate.

In the present embodiment, the input interface 137 is not limited to an input interface including a physical operational component such as a mouse, a keyboard, a trackball, a switch, a button, a joystick, a touch pad, or a touch panel display. For example, electrical signal processing circuitry that receives an electrical signal corresponding to an input operation from an external input device provided separately from the apparatus, and outputs the received electrical signal to the system control unit 11 is also an example of the input interface 137. The input interface 137 may include a tablet terminal or the like that can wirelessly communicate with the system control unit 11.

The memory 143 is implemented, for example, by a storage device such as an HDD, a solid state drive (SSD), or an integrated circuitry storage device that stores various types of information. Aside from the HDD, the SSD, and the like, the memory 143 may be implemented by a drive device that reads and writes various types of information from and into a portable storage medium such as a compact disc (CD), a digital versatile disc (DVD), or a flash memory, a semiconductor memory element such as a random access memory (RAM), or the like.

The memory 143 stores position information of various members in the X-ray diagnostic apparatus main body 110 at the time of execution of X-ray image capturing and X-ray fluoroscopy of the subject P. The position information of various members includes, for example, positions of the plurality of collimator blades of the X-ray collimator 1b, a position of the couch 5 with respect to the X-ray tube 1a, and the like. The position information is updated as appropriate in accordance with the X-ray image capturing and the X-ray fluoroscopy, for example, in accordance with the generation of an X-ray image. In addition, the memory 143 stores various programs for implementing the image processing function 111 and the transmission function 113.

The medical information display apparatus 150 includes the AR glasses 151 and processing circuitry 153. The AR glasses 151 correspond, for example, to a transmissive display. More specifically, the AR glasses 151 include a transmissive display area, and is capable of displaying medical information in the display area. The display area is set, for example in a central portion of a lens of the AR glasses 151. The AR glasses 151 are configured to be capable of displaying various types of medical information superimposed on a transmissive image, using a display control function 157 of the processing circuitry 153. Because a known configuration can be applied as the configuration of the AR glasses 151, the description thereof will be omitted.

The processing circuitry 153 includes an acquisition function 155 and the display control function 157. By reading out and executing the acquisition function 155 and the display control function 157 stored in a memory (not illustrated), the processing circuitry 153 implements various types of processing executed by the acquisition function 155 and the display control function 157.

The acquisition function 155 acquires medical information from the medical image collection apparatus (X-ray diagnostic apparatus) 110. Hereinafter, for the sake of clarity, the medical information is assumed to be a medical image (fluoroscopic image and/or captured image) generated by the X-ray diagnostic apparatus 110. A case where another type of medical information is acquired will be described in the modified examples. The acquisition function 155 acquires a fluoroscopic image generated through fluoroscopy executed by the X-ray diagnostic apparatus 110. In addition, the acquisition function 155 acquires a captured image generated by image capturing executed by the X- ray diagnostic apparatus 110. The acquisition function 155 stores the acquired fluoroscopic image and the captured image in a memory (not illustrated) of the medical information display apparatus 150. The processing circuitry 153 that implements the acquisition function 155 corresponds to an acquisition unit.

The display control function 157 changes a transparency of a medical information image indicating medical information in accordance with an operation performed on the X-ray diagnostic apparatus 110. For example, when a fluoroscopic image or a captured image is acquired in accordance with the operation performed on the X-ray diagnostic apparatus 110, the display control function 157 reduces the transparency of the fluoroscopic image or the captured image on the AR glasses 151 from a predetermined transparency to a set transparency (hereinafter referred to as a set transparency). The predetermined transparency is, for example, a transparency with which an object positioned behind the medical information is visually recognizable on the AR glasses 151. More specifically, a display target displayed in the display area of the AR glasses 151 is preliminarily displayed on the AR glasses 151 at a predetermined transparency. The set transparency is, for example, a transparency at which an object positioned behind the medical information is not visible on the AR glasses 151. The predetermined transparency and the set transparency are preliminary set and stored in a memory of the processing circuitry 153.

The display control function 157 may increase an opacity of the acquired fluoroscopic image or the captured image. The display control function 157 displays the fluoroscopic image or the captured image with reduced transparency in the display area of the AR glasses 151. The processing circuitry 153 that implements the display control function 157 corresponds to a display control unit.

Processing circuitry that implements the mechanism control unit 7, the system control unit 11, and the like described above, and the processing circuitry 153 include, as hardware resources, a processor such as a central processing unit (CPU), a micro processing unit (MPU), or graphics processing unit (GPU), and a memory such as a read only memory (ROM) and a RAM. In addition, the processing circuitry may be implemented by a processor such as an application specific integrated circuit (ASIC), a field programmable gate array (FPGA), another complex programmable logic device (CPLD), or a simple programmable logic device (SPLD).

Various functions to be executed by the processor are stored in a memory (not illustrated) in the form of a computer-executable program. The processor implements a function corresponding to each program by reading out and executing the corresponding program from the memory. In other words, each piece of circuitry in a state in which each program has been read out has a function corresponding to the read-out program.

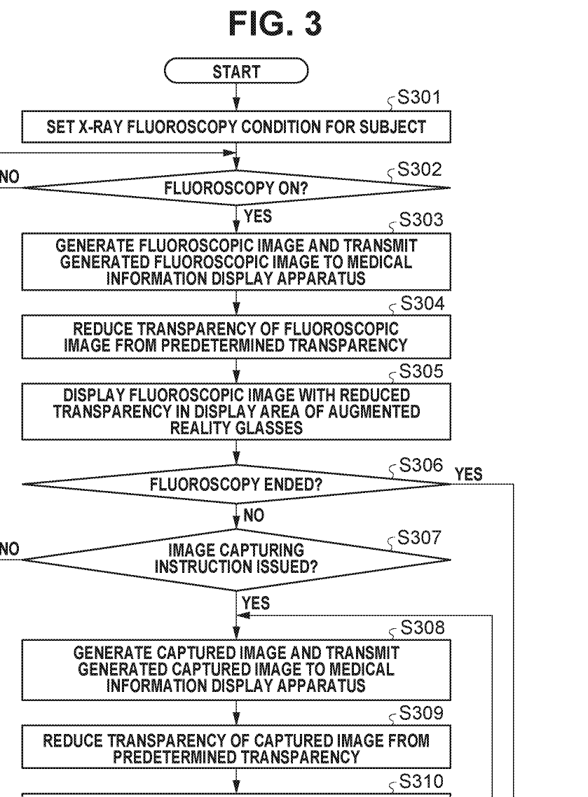

Heretofore, an overall configuration of the medical image collection system 100 according to an embodiment has been described. With the configuration, the medical information display apparatus 150 according to the embodiment executes processing of changing the transparency of medical information in accordance with an operation performed on the X-ray diagnostic apparatus 110 and displaying the medical information with the changed transparency in a display area of the AR glasses 151 (hereinafter referred to as medical information display processing). Hereinafter, a procedure for the medical information display processing will be described with reference to FIGS. 3 to 5.

FIG. 3 is a flowchart illustrating an example of the procedure for the medical information display processing. In the medical information display processing, an example where X-ray image capturing is executed during execution of X-ray fluoroscopy of the subject P and then X-ray fluoroscopy is executed again will be described.

### Medical Information Display Processing

### Step S301

Prior to the execution of X-ray fluoroscopy of the subject P, a condition for the X-ray fluoroscopy is set via the input interface 137. At this time, the subject P is placed on the couch 5. Further, positioning of the subject P with regard to the X-ray fluoroscopy is executed.

### Step S302

If the X-ray fluoroscopy is executed in accordance with an instruction issued by the user via the input interface 137 (YES in step S302), the processing in step S303 is executed. The execution of the X-ray fluoroscopy corresponds to, for example, turning fluoroscopy ON by a press of a foot switch related to fluoroscopy or a press of a fluoroscopy button. If the X-ray fluoroscopy is not executed (NO in step S302), the processing in step S302 is repeated.

### Step S303

The image data generation unit 3b generates a fluoroscopic image. The transmission function 113 transmits the generated fluoroscopic image to the medical information display apparatus 150. The fluoroscopic image to be transmitted to the medical information display apparatus 150 may be a fluoroscopic image on which image processing has been executed by the image processing function 111. The acquisition function 155 acquires the fluoroscopic image from the X-ray diagnostic apparatus 110.

### Step S304

The display control function 157 changes the transparency of the fluoroscopic image in accordance with an operation for turning fluoroscopy ON performed on the X-ray diagnostic apparatus 110. Specifically, the display control function 157 reduces the transparency of the acquired fluoroscopic image in accordance with a fluoroscopy execution instruction (fluoroscopy ON) from a transparency in the display area that had been set before the fluoroscopy ON.

### (Step S305)

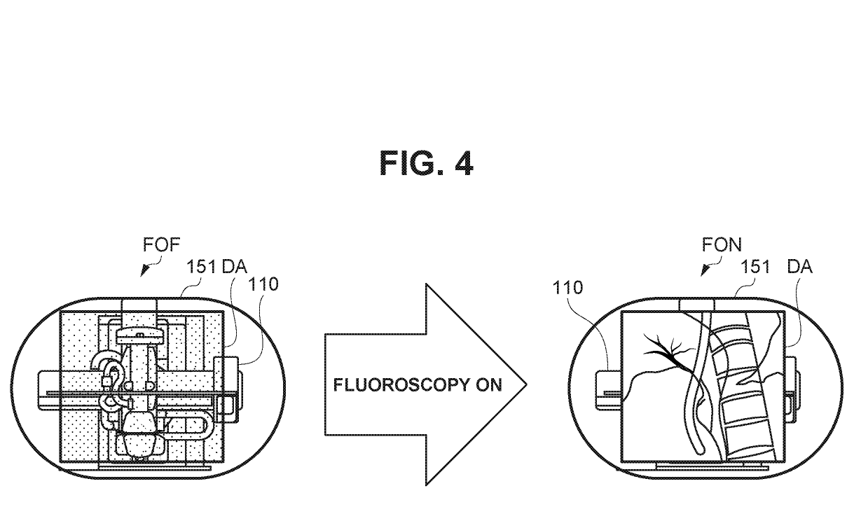

The display control function 157 displays the fluoroscopic image with the reduced transparency in the display area of the AR glasses 151. FIG. 4 is a diagram illustrating an example of medical information displayed on the AR glasses 151 before and after the fluoroscopy ON. As illustrated in FIG. 4, before fluoroscopy is turned ON (FOF), the user can visually recognize the X-ray diagnostic apparatus main body 110 through the transmissive AR glasses 151. At this time, in a display area DA, the X-ray diagnostic apparatus main body 110 is displayed with a predetermined transparency. The transparency of the display area DA before fluoroscopy is turned ON (FOF) may be maximum.

As illustrated in FIG. 4, in response to the fluoroscopy ON, a fluoroscopic image with a reduced transparency is displayed in the display area DA of the AR glasses 151 (FON). Accordingly, the user wearing the medical information display apparatus 150 can easily recognize the fluoroscopic image.

### Step S306

If fluoroscopy of the subject P is ended (YES in step S306), the medical information display processing ends. The end of fluoroscopy of the subject P corresponds to, for example, a release of the press of the foot switch related to fluoroscopy, a release of the press of the fluoroscopy button, or the like. If the fluoroscopy of the subject P is not ended (NO in step S306), the processing in step S307 is executed.

### Step S307

If an instruction of image capturing (X-ray image capturing) of the subject P is issued (YES in step S307), the processing in step S308 is executed. The instruction of X-ray image capturing corresponds to, for example, a press of a foot switch related to image capturing, a press of an image capturing button, or the like. The instruction of X-ray image capturing may also be referred to as image capturing ON. If an instruction of image capturing (X-ray image capturing) of the subject P is not issued (NO in step S307), the processing in step S302 and subsequent steps is repeated.

### Step S308

The image data generation unit 3b generates a captured image. The transmission function 113 transmits the generated captured image to the medical information display apparatus 150. The captured image to be transmitted to the medical information display apparatus 150 may be a captured image on which image processing has been executed by the image processing function 111. The acquisition function 155 acquires the captured image from the X-ray diagnostic apparatus 110.

### Step S309

The display control function 157 changes the transparency of the captured image in accordance with an operation for turning image capturing ON performed on the X-ray diagnostic apparatus 110. Specifically, the display control function 157 reduces the transparency of the acquired captured image in accordance with an image capturing execution instruction (image capturing ON) from a transparency in the display area that had been set before the image capturing ON.

### Step S310

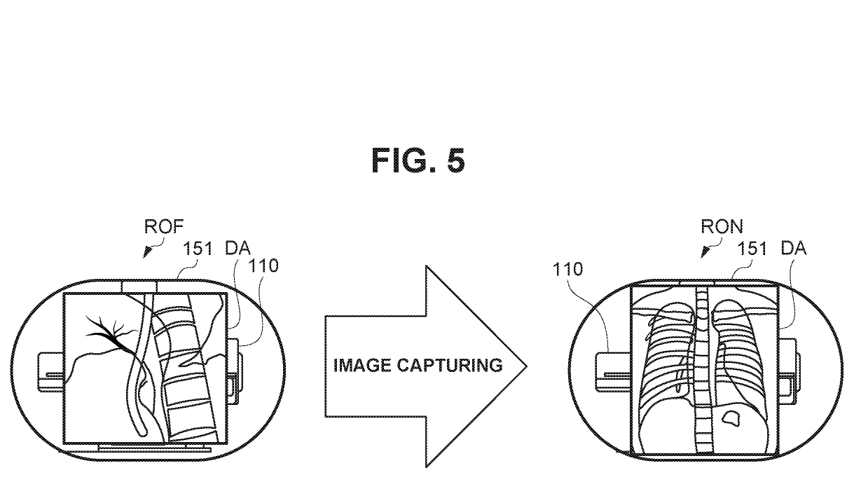

The display control function 157 displays the captured image with the reduced transparency in the display area of the AR glasses 151. FIG. 5 is a diagram illustrating an example of medical information displayed on the AR glasses 151 before and after the image capturing ON. As illustrated in FIG. 5, before image capturing is turned ON (ROF), the user can visually recognize a fluoroscopic image through the transmissive AR glasses 151. At this time, in the display area DA, the fluoroscopic image is displayed.

As illustrated in FIG. 5, in response to the image capturing ON, a captured image with a reduced transparency is displayed in the display area DA of the AR glasses 151 (RON). Accordingly, the user wearing the medical information display apparatus 150 can easily recognize the captured image.

### STEP S311

If image capturing of the subject P is ended (YES in step S311), the processing in step S302 and subsequent steps is repeated. The end of image capturing of the subject P corresponds to, for example, a release of the press of the foot switch related to image capturing, a release of the press of the image capturing button, or the like. If the image capturing of the subject P is not ended (NO in step S311), the processing in step S308 and subsequent steps is executed.

The medical information display apparatus 150 according to the embodiment that has been described above includes the transmissive display area DA and the AR glasses 151 that can display medical information in the display area DA, acquires medical information from the medical image collection apparatus 110, changes the transparency of a medical information image indicating the medical information, in accordance with an operation performed on the medical image collection apparatus 110, and displays the medical information image with the changed transparency in the display area DA. For example, regarding the medical information display apparatus 150 according to the embodiment, the operation performed on the medical image collection apparatus 110 is an operation related to the collection of medical images that is performed by the medical image collection apparatus 110, and the medical information corresponds to medical images collected by the medical image collection apparatus 110.

Specifically, regarding the medical information display apparatus 150 according to the embodiment, the medical image collection apparatus is the X-ray diagnostic apparatus 110, the operation is an execution instruction of fluoroscopy of the subject P, the medical information is a fluoroscopic image generated by the fluoroscopy, and the medical information display apparatus 150 according to the embodiment reduces the transparency of the fluoroscopic image in accordance with the fluoroscopy execution instruction and displays the fluoroscopic image with the reduced transparency in the display area DA. In addition, regarding the medical information display apparatus 150 according to the embodiment, the medical image collection apparatus is the X-ray diagnostic apparatus 110, the operation is an instruction of image capturing of the subject P, the medical information is a captured image generated by the image capturing, and the medical information display apparatus 150 according to the embodiment reduces the transparency of the captured image in accordance with the image capturing instruction and displays the captured image with the reduced transparency in the display area DA.

In view of the foregoing, with the medical information display apparatus 150 according to the embodiment, it is possible to display a fluoroscopic image and a captured image on the AR glasses 151 after reducing transparencies of the fluoroscopic image and the captured image (i.e., increasing non-transparencies of the fluoroscopic image and the captured image) in accordance with a fluoroscopy ON and an image capturing ON. Because the user desires a fluoroscopic image and a captured image as medical information in the operations for turning fluoroscopy ON and image capturing ON, with the medical information display apparatus 150 according to the embodiment, it is possible to display the medical information desired by the user on the AR glasses 151 with an appropriate transparency or nontransparency.

In view of the foregoing, with the medical information display apparatus 150 according to the embodiment, on the AR glasses 151, it is possible to appropriately change the transparency (nontransparency) related to the display of medical information in accordance with a usage scene of a medical device. Accordingly, with the medical information display apparatus 150 according to the embodiment, it is possible to provide medical information depending on a usage scene of a medical device without causing inconvenience to the user and without performing various types of adjustment related to the display of medical information on the AR glasses 151. Thus, with the medical information display apparatus 150 according to the embodiment, it is possible to improve the visibility regarding the display of medical information on the AR glasses 151 and improve the diagnostic efficiency (throughput) for the user.

### First Modified Example

In a first modified example, as a trigger for displaying a medical image with a reduced transparency on the AR glasses 151, an action of the user wearing the AR glasses 151 may be used in some cases. The action of the user corresponds to, for example, an action related to a direction of a line of sight of the user with respect to the X-ray diagnostic apparatus 110, the orientation of the head of the user, or the like. Accordingly, the medical information display apparatus 150 according to the present modified example reduces the transparency of medical information displayed in the display area DA of the AR glasses 151 in accordance with the line of sight of the user with respect to the X-ray diagnostic apparatus 110 or the orientation of the head of the user, for example, during execution of fluoroscopy of the subject P,. Hereinafter, a configuration and processing contents of the present modified example will be described.

The medical information display apparatus 150 further includes a sensor that detects a line of sight of the user or the orientation of the head of the user. The sensor that detects the line of sight of the user (hereinafter referred to as an eye-gaze sensor) detects a line of sight of the user wearing the AR glasses 151, with the position of the X-ray diagnostic apparatus 110 as a reference. The eye-gaze sensor outputs the detected line of sight of the user to the processing circuitry 153. The sensor that detects the orientation of the head of the user (hereinafter referred to as a head direction sensor) detects the orientation of the head of the user wearing the AR glasses 151, with the position of the X-ray diagnostic apparatus 110 as a reference. The head direction sensor outputs the detected orientation of the head of the user to the processing circuitry 153. Because known sensors can be used as the eye-gaze sensor and the head direction sensor, the description thereof will be omitted.

Using the display control function 157, the processing circuitry 153 determines whether the line of sight of the user or the orientation of the head of the user is apart from the X-ray diagnostic apparatus 110 by a predetermined distance, for example, during the execution of fluoroscopy of the subject P. Whether the fluoroscopy of the subject P is being executed corresponds to whether fluoroscopic images are acquired by the acquisition function 155. If fluoroscopic images are acquired at a predetermined time interval (for example, pulses of X-ray radiation), the display control function 157 determines that fluoroscopy of the subject P is being executed.

The predetermined distance is preset and stored in a memory in the processing circuitry 153. The determination corresponds to the user looking away from the X-ray diagnostic apparatus 110. When it is determined that the user is looking away, the display control function 157 reduces the transparency of the latest fluoroscopic image acquired. Because the reduction of the transparency of the fluoroscopic image is similar to that in the embodiment, the description thereof will be omitted. The display control function 157 displays a fluoroscopic image with a reduced transparency in the display area DA of the AR glasses 151.

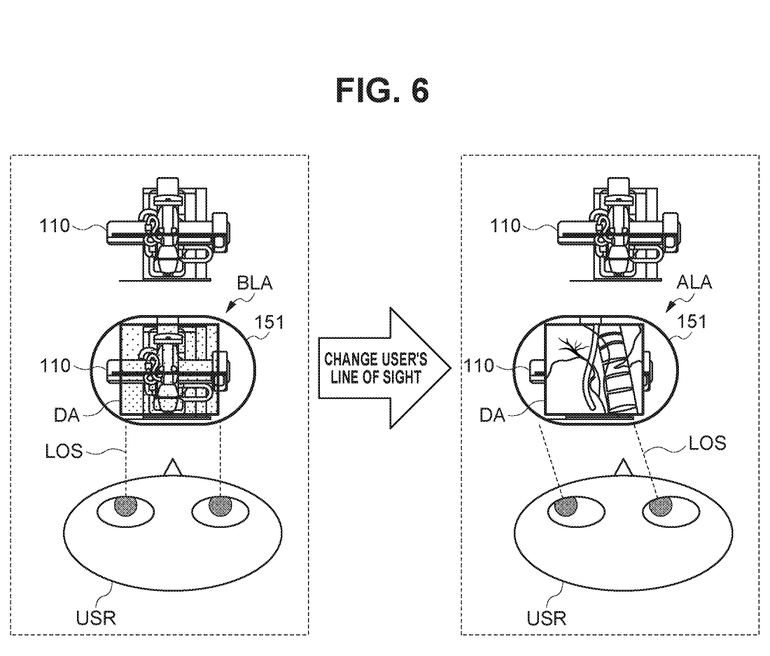

FIG. 6 is a diagram illustrating an example of a fluoroscopic image displayed on the AR glasses 151 before and after a change of a line of sight LOS of a user USR. As illustrated in FIG. 6, during fluoroscopy, if the line of sight LOS of the user USR is directed toward the X-ray diagnostic apparatus 110, i.e., before a line of sight change BLA, a fluoroscopic image is displayed with a predetermined transparency in the display area DA of the AR glasses 151. At this time, a fluoroscopic image may not be displayed in the display area DA of the AR glasses 151. During fluoroscopy, if the line of sight LOS of the user USR is directed toward the X-ray diagnostic apparatus 110, i.e., after a line of sight change ALA, the user USR can visually recognize the X-ray diagnostic apparatus 110 through the AR glasses 151 as illustrated in FIG. 6. On the other hand, as illustrated in FIG. 6, if the line of sight LOS of the user USR deviates from the X-ray diagnostic apparatus 110, a fluoroscopic image with a reduced transparency is displayed on the AR glasses 151.

In the above-described medical information display apparatus 150 according to the first modified example of the embodiment, the medical image collection apparatus is the X-ray diagnostic apparatus 110, an operation performed on the medical image collection apparatus 110 corresponds to the line of sight LOS of the user USR wearing the AR glasses 151 and/or the front side of the head of the user USR deviating from the X-ray diagnostic apparatus 110 during execution of fluoroscopy of the subject P, and medical information displayed on the AR glasses 151 is a fluoroscopic image generated by fluoroscopy of the subject P. The medical information display apparatus 150 according to the first modified example reduces transparency of the fluoroscopic image in accordance with the line of sight LOS and/or the front side of the head deviating from the X-ray diagnostic apparatus 110 during the execution of the fluoroscopy, and displays the fluoroscopic image with the reduced transparency on the display area DA.

Accordingly, with the medical information display apparatus 150 according to the first modified example, the user USR wearing the medical information display apparatus 150 can easily recognize a fluoroscopic image in response to an action of the user USR with respect to the medical image collection apparatus 110. Because other effects are included in the embodiment, the description thereof will be omitted.

### Second Modified Example

Medical information according to a second modified example is a medical image collected by a medical device related to the medical image collection apparatus 110. The medical information display apparatus 150 according to the present modified example displays a medical image with a reduced transparency in the display area DA of the AR glasses 151, for example, with turning OFF of the fluoroscopy of the subject P as a trigger. The medical device according to the present modified example is an image collection device that can collect images of the subject P from inside of the subject P, for example, in the fluoroscopy of the subject P. At this time, the medical image corresponds to an internal image of the subject, which is an image of the subject P collected from the inside of the subject P.

The image collection device is, for example, an endoscope, an ultrasonic diagnostic apparatus including an ultrasonic probe, or the like. The endoscope may include an ultrasonic probe. In a case where the image collection device is an endoscope, the internal image of the subject corresponds to an endoscopic image. Further, in a case where the image collection device is an ultrasonic diagnostic apparatus, the internal image of the subject corresponds to an ultrasonic image. The ultrasonic probe used in the ultrasonic diagnostic apparatus is, for example, a transesophageal probe, an intravascular ultrasound catheter (intravascular probe), or the like.

In a case where the image collection device is an endoscope, a medical image displayed in the display area DA of the AR glasses 151 is an endoscopic image. In addition, in a case where the image collection device is an ultrasonic diagnostic apparatus, a medical image displayed in the display area DA of the AR glasses 151 is an ultrasonic image. In addition, in a case where the image collection device is an endoscope including an ultrasonic probe, a medical image displayed in the display area DA of the AR glasses 151 is an endoscopic image or an ultrasonic image.

The present modified example is implemented in examinations of endoscopic retrograde cholangiopancreatography (ERCP), transesophageal echocardiogram (TEE), intravascular ultrasound (IVUS), and the like, for example. Hereinafter, for the sake of clarity, it is assumed that the image collection device is an endoscope, and the medical image is an endoscopic image. In addition, in a case where the IVUS is applied as the present modified example, the medical image collection apparatus 110 corresponds to, for example, an X-ray angiography apparatus.

The processing circuitry 153 acquires an endoscopic image from the endoscope using the acquisition function 155. The acquisition function 155 stores the acquired endoscopic image in a memory. In addition, the acquisition function 155 acquires a stop instruction of fluoroscopy of the subject P from the X-ray diagnostic apparatus 110.

Using the display control function 157, the processing circuitry 153 reduces the transparency of the endoscopic image with the acquisition of the fluoroscopy stop instruction as a trigger, i.e., in accordance with the fluoroscopy stop instruction. Because a reduction of the transparency of the endoscopic image is similar to that in the embodiment, the description thereof will be omitted. Subsequently, the display control function 157 displays the endoscopic image with the reduced transparency in the display area DA of the AR glasses 151.

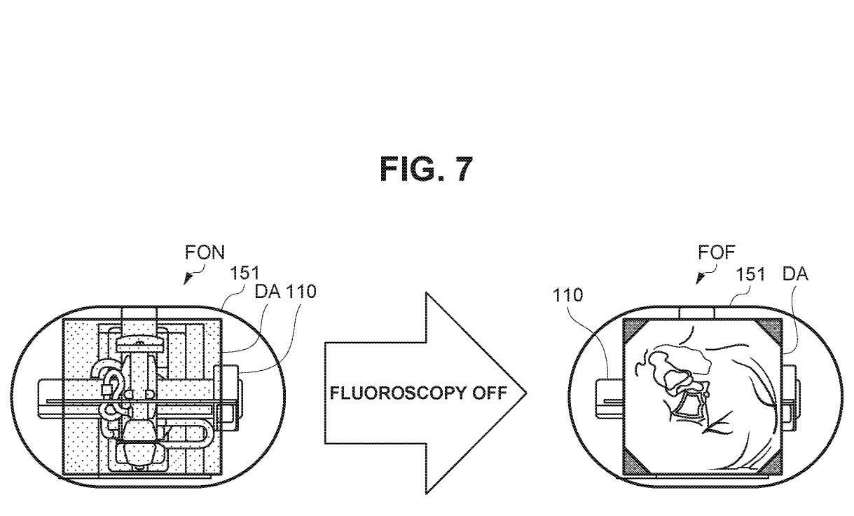

FIG. 7 is a diagram illustrating an example of medical information displayed on the AR glasses 151 before and after a fluoroscopy OFF. As illustrated in FIG. 7, before fluoroscopy is turned OFF (FON), the user can visually recognize the X-ray diagnostic apparatus main body 110 through the transmissive AR glasses 151. At this time, in the display area DA, the X-ray diagnostic apparatus main body 110 is displayed with a predetermined transparency. Before the fluoroscopy is turned OFF (FON), a fluoroscopic image with a reduced transparency may be displayed in the display area DA. At this time, the medical information display processing according to the present modified example is executed as processing to be executed in a case where it is determined that fluoroscopy of the subject P is ended (YES in step S306).

Further, as illustrated in FIG. 7, in response to the fluoroscopy OFF, an endoscopic image with a reduced transparency is displayed in the display area DA of the AR glasses 151 (FOF). Accordingly, the user wearing the medical information display apparatus 150 can easily recognize the endoscopic image.

In the above-described medical information display apparatus 150 according to the second modified example of the embodiment, the medical image collection apparatus is the X-ray diagnostic apparatus 110, an operation performed on the medical image collection apparatus 110 is a stop instruction of fluoroscopy of the subject P, the medical device is an image collection device capable of collecting images of the subject P from the inside of the subject P. The medical information display apparatus 150 according to the second modified example acquires the fluoroscopy stop instruction, reduces the transparency of an internal image of the subject in accordance with the fluoroscopy stop instruction, and displays the internal image of the subject with the reduced transparency in the display area DA.

Accordingly, with the medical information display apparatus 150 according to the second modified example, the user USR wearing the medical information display apparatus 150 can easily visually recognize a medical image (endoscopic image and/or ultrasonic image) acquired from the medical device in response to termination of the fluoroscopy. Because other effects are included in the embodiment, the description thereof will be omitted.

### Third Modified Example

In a third modified example, brightness of a region outside a fluoroscopic image in the display area DA (hereinafter referred to as an external region) is made darker than that of the fluoroscopic image in accordance with a fluoroscopy execution instruction. More specifically, in the present modified example, the display control function 157 makes the brightness of the external region of the display area DA, i.e., the brightness of a periphery (may also be referred to as a peripheral region) of the display area DA, on the AR glasses 151 darker than that of the fluoroscopic image displayed on the AR glasses 151, in accordance with a fluoroscopy ON. The dimming is not limited to, for example, making the brightness of the external region darker than that of the fluoroscopic image, and may be implemented by closing a shutter provided in the AR glasses 151, i.e., by shutting out light transmitted from the outside through the AR glasses 151.

In addition, the dimming has been described above as being executed in accordance with the fluoroscopy ON, but this is not limiting. The display control function 157 may make the brightness of the external region darker than that of a captured image in accordance with an image capturing ON. In addition, the display control function 157 may make the brightness of the external region darker than that of a medical image (internal image of the subject) acquired from a medical device in accordance with a fluoroscopy OFF.

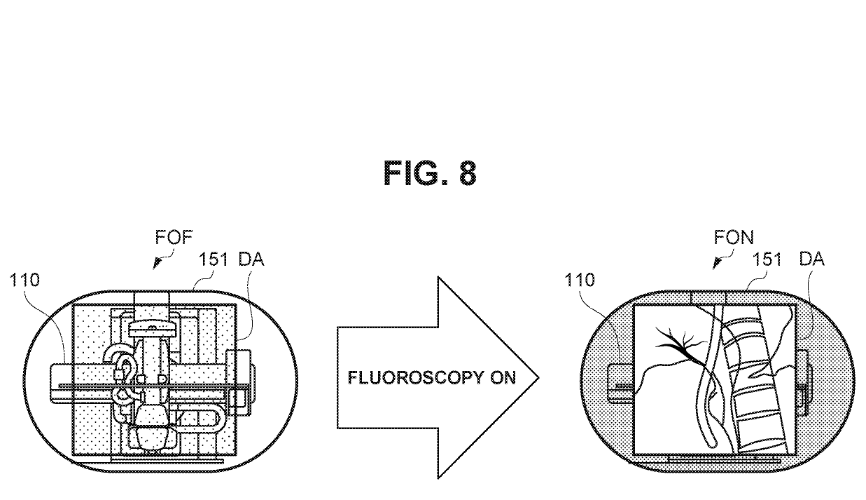

FIG. 8 is a diagram illustrating an example of medical information displayed on the AR glasses 151 before and after a fluoroscopy ON. As illustrated in FIG. 8, before fluoroscopy is turned ON (FOF), the user can visually recognize the X-ray diagnostic apparatus main body 110 through the transmissive AR glasses 151. As illustrated in FIG. 8, in response to the fluoroscopy ON, a fluoroscopic image with a reduced transparency is displayed in the display area DA of the AR glasses 151 (FON). At this time, as illustrated in FIG. 8, the brightness of the external region of the display area DA is darker than the brightness of the fluoroscopic image.

The above-described medical information display apparatus 150 according to the third modified example of the embodiment makes the brightness of an external region of a fluoroscopic image in the display area DA darker than that of the fluoroscopic image in accordance with a fluoroscopy execution instruction. In addition, the medical information display apparatus 150 according to the third modified example of the embodiment makes the brightness of an external region of a captured image in the display area DA darker than that of the captured image in accordance with an execution instruction of image capturing. In addition, the medical information display apparatus 150 according to the third modified example of the embodiment makes the brightness of an external region of an internal image of the subject in the display area DA darker than that of the internal image of the subject in accordance with a fluoroscopy stop instruction.

Accordingly, with the medical information display apparatus 150 according to the third modified example, the user USR wearing the medical information display apparatus 150 can more easily visually recognize a medical image displayed in the display area DA. Because other effects are included in the embodiment, the description thereof will be omitted.

### Fourth Modified Example

A fourth modified example uses collection information related to collection of medical images as the medical information displayed in the display area DA of the AR glasses 151. The collection information is, for example, a pictogram indicating that X-rays are being emitted (hereinafter referred to as an exposure pictogram). The pictogram as the collection information is not limited to the exposure pictogram, and may also be another pictogram indicating an operation of the X-ray diagnostic apparatus 110 (for example, movement of the couch 5, or the like).

The display control function 157 reduces transparency of an irradiation pictogram indicating X-ray irradiation in the display area DA in accordance with an execution instruction of fluoroscopy or image capturing of the subject P. Because a reduction of the transparency of the irradiation pictogram is similar to that in the embodiment, the description thereof will be omitted. Subsequently, the display control function 157 displays the irradiation pictogram with the reduced transparency in the display area DA of the AR glasses 151.

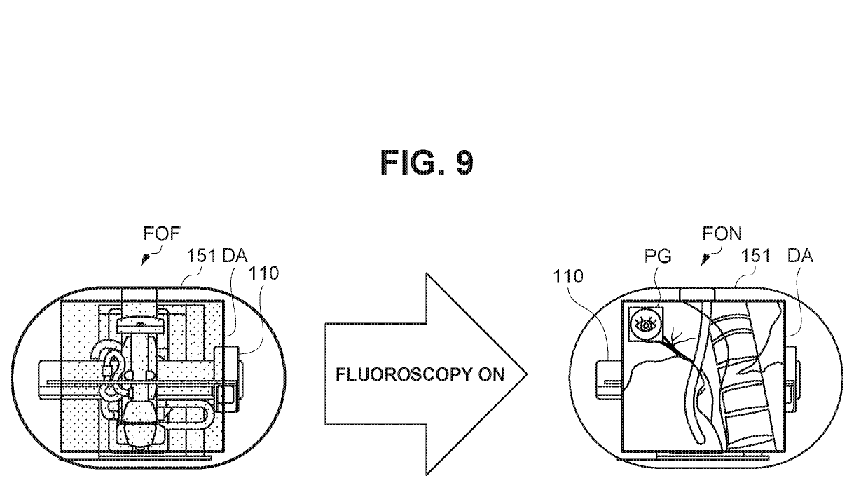

FIG. 9 is a diagram illustrating an example of medical information displayed on the AR glasses 151 before and after a fluoroscopy ON. As illustrated in FIG. 9, before fluoroscopy is turned ON (FOF), the user can visually recognize the X-ray diagnostic apparatus main body 110 through the transmissive AR glasses 151. As illustrated in FIG. 9, in response to the fluoroscopy ON, an exposure pictogram PG with a reduced transparency is displayed in the display area DA of the AR glasses 151 together with a fluoroscopic image (FON).

In the above-described medical information display apparatus 150 according to the fourth modified example of the embodiment, the medical image collection apparatus is the X-ray diagnostic apparatus 110, the operation performed on the medical image collection apparatus 110 is an instruction of fluoroscopy or image capturing of the subject P, medical information acquired from the medical image collection apparatus 110 is a fluoroscopic image generated by fluoroscopy or a captured image generated by image capturing, and the collection information related to the collection of medical images is a pictogram indicating that X-rays are being emitted. The medical information display apparatus 150 according to the fourth modified example reduces the transparency of the pictogram and the transparency of the fluoroscopic image or the captured image in accordance with the instruction of fluoroscopy or image capturing, and displays the pictogram with the reduced transparency in the display area DA together with the fluoroscopic image or the captured image with the reduced transparency.

Accordingly, with the medical information display apparatus 150 according to the fourth modified example, the user USR (e.g., a technician) wearing the medical information display apparatus 150 can easily grasp, for example, an operation of the medical image collection apparatus 110. Because other effects are included in the embodiment, the description thereof will be omitted.

### Fifth Modified Example

A fifth modified example uses vital signs of the subject P that are related to a medical image, as collection information, as the medical information displayed in the display area DA of the AR glasses 151. The vital signs include, for example, blood pressure, pulse rate, respiratory rate, body temperature, and the like.

The acquisition function 155 acquires the vital signs from the X-ray diagnostic apparatus 110 or a vital signs monitor. The display control function 157 reduces the transparency of an image indicating the vital signs, in the display area DA in accordance with an execution instruction of fluoroscopy or image capturing of the subject P. Because a reduction of the transparency of the image indicating the vital signs is similar to that in the embodiment, the description thereof will be omitted. Subsequently, the display control function 157 displays the vital signs with a reduced transparency in the display area DA of the AR glasses 151.

In the above-described medical information display apparatus 150 according to the fifth modified example of the embodiment, the medical image collection apparatus is the X-ray diagnostic apparatus 110, an operation performed on the medical image collection apparatus 110 is an instruction of fluoroscopy or image capturing of the subject P, medical information acquired from the medical image collection apparatus 110 is a fluoroscopic image generated by fluoroscopy or a captured image generated by image capturing, and the collection information related to the collection of medical images is vital signs of the subject P that are related to medical images. The medical information display apparatus 150 according to the fifth modified example reduces the transparency of an image indicating the vital signs and the transparency of the fluoroscopic image or the captured image in accordance with the instruction of fluoroscopy or image capturing, and displays the image indicating the vital signs with the reduced transparency in the display area DA together with the fluoroscopic image or the captured image with the reduced transparency.

Accordingly, with the medical information display apparatus 150 according to the fifth modified example, the user USR (e.g., a nurse) wearing the medical information display apparatus 150 can easily grasp the vital signs of the subject P. Because other effects are included in the embodiment, the description will be omitted.

### Sixth Modified Example

A sixth modified example uses X-ray irradiation information related to the collection of medical images as the medical information displayed in the display area DA of the AR glasses 151. The X-ray irradiation information includes, for example, an X-ray cone, a field of view, an arrangement of a compensating filter, a cumulative exposure dose to the subject P (for example, entrance skin dose), and the like.

The acquisition function 155 acquires the X-ray irradiation information from the X-ray diagnostic apparatus 110. The display control function 157 reduces the transparency of an image indicating the X-ray irradiation information in accordance with an execution instruction of fluoroscopy or image capturing of the subject P. Because a reduction of the transparency of the image indicating the X-ray irradiation information is similar to that in the embodiment, the description thereof will be omitted. Subsequently, the display control function 157 displays the X-ray irradiation information with the reduced transparency in the display area DA of the AR glasses 151. In a case where the X-ray irradiation information is a cumulative exposure dose, the display control function 157 may display the X-ray irradiation information by varying the hue in accordance with the value of the cumulative exposure dose, using a plurality of preset threshold values, in the display area DA of the AR glasses 151.

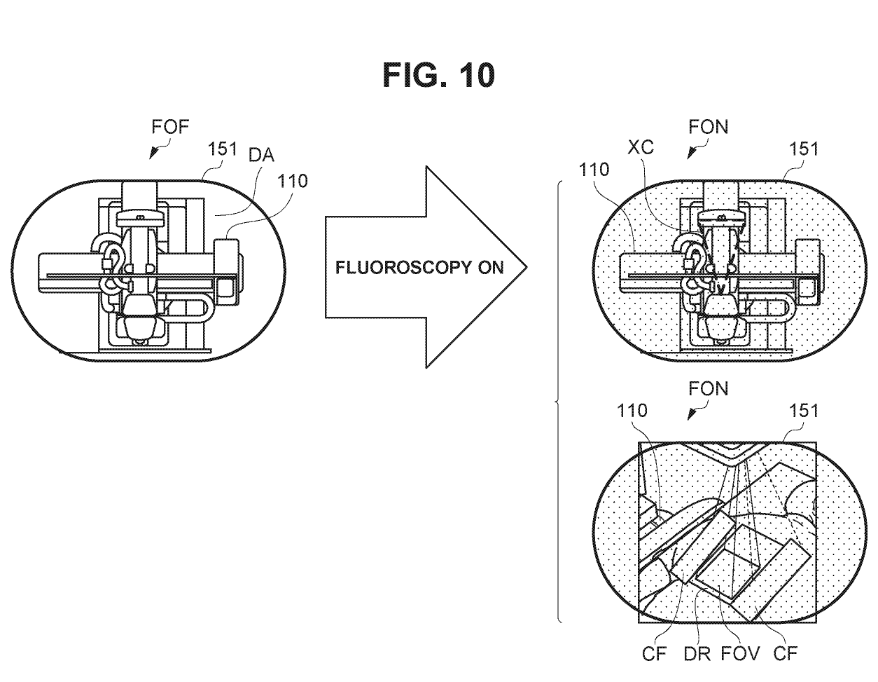

FIG. 10 is a diagram illustrating an example of medical information displayed on the AR glasses 151 before and after a fluoroscopy ON. As illustrated in FIG. 10, before fluoroscopy is turned ON (FOF), the user can visually recognize the X-ray diagnostic apparatus main body 110 through the transmissive AR glasses 151. As illustrated in FIG. 10, in response to the fluoroscopy ON, an X-ray cone XC is displayed on the AR glasses 151. In addition, as another example, as illustrated in FIG. 10, in response to the fluoroscopy ON, a field of view FOV, a position CF of the compensating filter, and an X-ray detection range DR in which X-rays are detected by the X-ray detection unit 3 are displayed on the AR glasses 151.

In the above-described medical information display apparatus 150 according to the sixth modified example of the embodiment, the medical image collection apparatus is the X-ray diagnostic apparatus 110, the operation performed on the medical image collection apparatus 110 is an instruction of fluoroscopy or image capturing of the subject P, collection information related to the collection of medical images is X-ray irradiation information related to emission of X-rays toward the subject P. The medical information display apparatus 150 according to the sixth modified example reduces the transparency of the image indicating the X-ray irradiation information in accordance with the instruction of fluoroscopy or image capturing, and displays the image indicating the X-ray irradiation information with the reduced transparency in the display area DA.

Accordingly, with the medical information display apparatus 150 according to the sixth modified example, the user USR (e.g., a technician, a doctor) wearing the medical information display apparatus 150 can easily grasp X-ray irradiation information related to the X-ray irradiation. Because other effects are included in the embodiment, the description thereof will be omitted.

### Seventh Modified Example

A seventh modified example reduces the transparency of an image indicating a movable range of a movable part in accordance with an instruction to move the movable part in the medical image collection apparatus 110, and displays the image on the AR glasses 151. The instruction to move the movable part in the medical image collection apparatus 110 is included in the operation related to the collection of medical images. In a case where the medical image collection apparatus is the X-ray diagnostic apparatus 110, the movable part is, for example, the support arm 4, the couch 5, and the like. At this time, the movable range corresponds to a movable range of the support arm 4 and a movable range of the couch 5.

The acquisition function 155 acquires the movable range of the movable part in the medical image collection apparatus 110 from the X-ray diagnostic apparatus 110. In addition, the acquisition function 155 acquires the instruction to move the movable part in the X-ray diagnostic apparatus 110 from the X-ray diagnostic apparatus 110.

The display control function 157 reduces the transparency of an image indicating the movable range corresponding to the movable part, in accordance with the instruction to move the movable part. Because a reduction of the transparency of the image indicating the movable range is similar to that in the embodiment, the description thereof will be omitted. Subsequently, the display control function 157 displays the image indicating the movable range with the reduced transparency on the AR glasses 151.

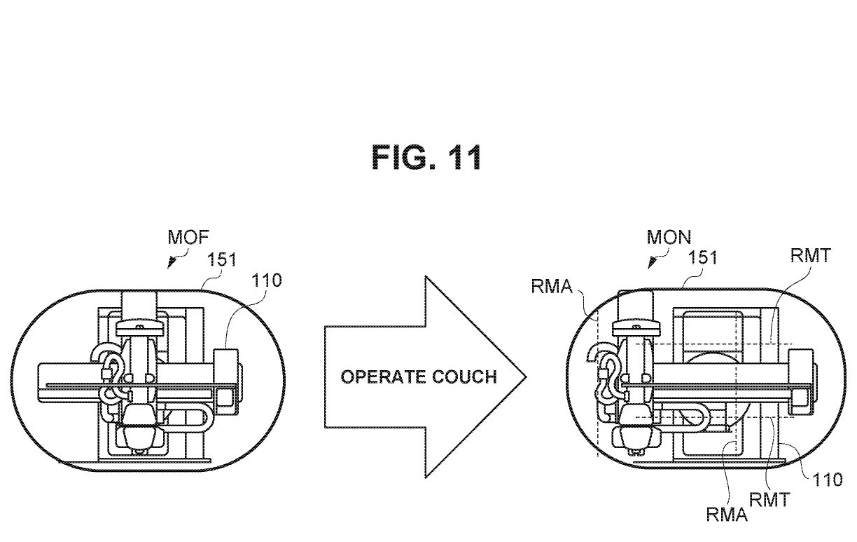

FIG. 11 is a diagram illustrating an example of medical information displayed on the AR glasses 151 before and after input of a moving operation of the couch 5. As illustrated in FIG. 11, before the moving operation of the couch 5 (MOF), the user can visually recognize the X-ray diagnostic apparatus main body 110 through the transmissive AR glasses 151. As illustrated in FIG. 11, in response to the input of the moving operation of the couch 5 (MON), a movable range RMT of the couch 5 is displayed on the AR glasses 151. At this time, in FIG. 11, a movable range RMA of the support arm 4 is also displayed on the AR glasses 151. A movable range may be displayed on the AR glasses 151 in accordance with a movable part that is a target of the moving operation. For example, in a case where a moving operation of the couch 5 is input, only the movable range RMT of the couch 5 is displayed on the AR glasses 151.

In the above-described medical information display apparatus 150 according to the seventh modified example of the embodiment, the medical image collection apparatus is the X-ray diagnostic apparatus 110, and an operation performed on the medical image collection apparatus 110 is an operation of a movable part in the medical image collection apparatus. The medical information display apparatus 150 according to the seventh modified example reduces the transparency of an image indicating a movable range in accordance with the instruction to move the movable part, and displays the image indicating the movable range with the reduced transparency on the AR glasses 151.

Accordingly, with the medical information display apparatus 150 according to the seventh modified example, the user USR (e.g., a technician, a doctor, a nurse) wearing the medical information display apparatus 150 can easily grasp a movable range of a movable part. Thus, with the medical information display apparatus 150 according to the seventh modified example, it is possible to reduce a possibility that the user USR touches the movable part in moving the movable part in the medical image collection apparatus 110, and improve the safety of the user USR. Because other effects are included in the embodiment, the description thereof will be omitted.

### Application Example

In the above-described embodiment and the first to seventh modified examples, processing of changing a transparency, and the like are performed in the medical information display apparatus 150. In an application example, however, the processing performed by the medical information display apparatus 150 according to the embodiment is executed by a medical image collection apparatus such as an X-ray diagnostic apparatus.

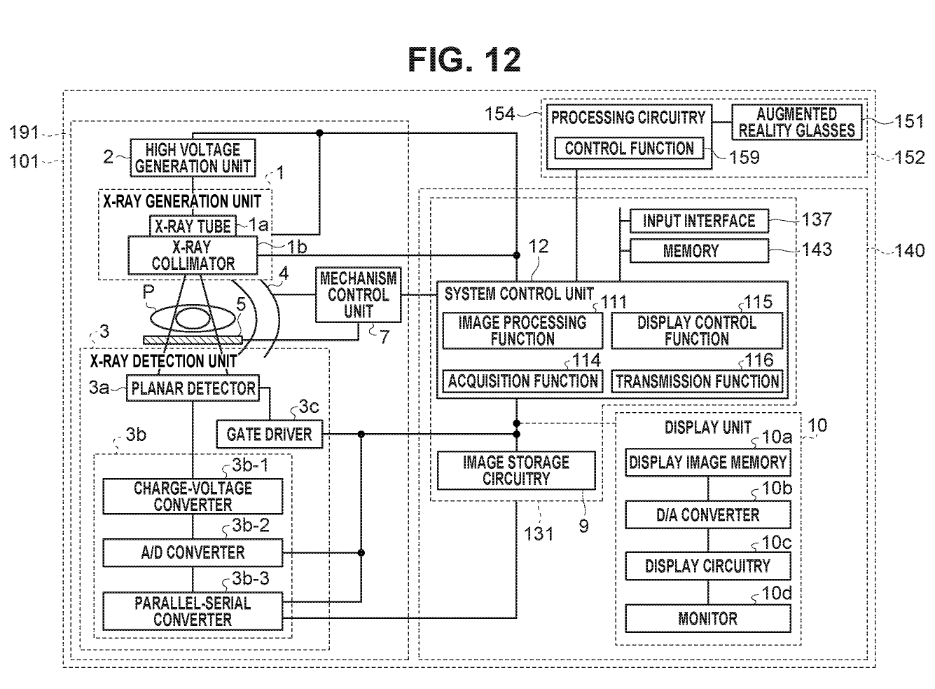

FIG. 12 is a diagram illustrating an example of a configuration of a medical image collection system 101 according to the application example. In FIG. 12, components that perform processing, operations, and the like that are similar to those of the components illustrated in FIG. 2 are indicated by reference numerals similar to the reference numerals in FIG. 2. Hereinafter, for the sake of clarity, the description will be provided assuming that a medical image collection apparatus 191 illustrated in FIG. 12 is an X-ray diagnostic apparatus. In addition, in the present application example, components different from those in the embodiment will be described.

The medical image collection system 101 includes an X-ray diagnostic apparatus main body (hereinafter referred to as an X-ray diagnostic apparatus) 191 implemented as a medical image collection apparatus, a console apparatus 140, and a medical information display apparatus 152. The medical information display apparatus 152 includes processing circuitry 154 and AR glasses 151. The processing circuitry 154 has a control function 159. The control function 159 receives medical information transmitted from the X-ray diagnostic apparatus 191 and the like, and a control signal output from a display control function 115 in a system control unit 12. The control function 159 displays the received medical information on the AR glasses 151 in accordance with the control signal received from the X-ray diagnostic apparatus 191. Because a display mode of the medical information displayed on the AR glasses 151 in the present application example is similar to that in the embodiment and the first to seventh modified examples, the description of the display mode of the medical information on the AR glasses 151 will be omitted.

The system control unit 12 in the X-ray diagnostic apparatus 191 includes the image processing function 111, an acquisition function 114, the display control function 115, and a transmission function 116. The acquisition function 114 acquires medical information to be displayed in the transmissive display area DA of the AR glasses 151. For example, the acquisition function 114 acquires, as the medical information, at least one of an X-ray image (fluoroscopic image, captured image) collected by the X-ray diagnostic apparatus 191, a medical image (endoscopic image, ultrasonic image, or the like) collected by a medical device (e.g., endoscope, ultrasonic diagnostic apparatus, or the like) related to the X-ray diagnostic apparatus 191, collection information related to the collection of medical images such as an X-ray image, an endoscopic image, and a ultrasonic image, and a movable range of a movable part in the X-ray diagnostic apparatus 191.

Specifically, the acquisition function 114 acquires an X-ray image (fluoroscopic image, captured image) generated by an image data generation unit 3b from the image data generation unit 3b as the medical information. The acquisition function 114 may acquire an X-ray image on which image processing has been executed by the image processing function 111 from the image processing function 111 as the medical information, or acquire an X-ray image stored in the image storage circuitry 9 as the medical information. In addition, the acquisition function 114 may acquire an action of the user wearing the AR glasses 151 from the medical information display apparatus 152. The system control unit 12 that implements the acquisition function 114 is referred to as an acquisition unit.

The display control function 115 changes the transparency of a medical information image indicating the medical information in accordance with an operation performed on the X-ray diagnostic apparatus 191. The operation is at least one of an operation related to the collection of medical images performed by the X-ray diagnostic apparatus 191 and an action of the user wearing the AR glasses 151. Because processing contents of a transparency change are similar to those in the embodiment and the first to seventh modified examples, the description thereof will be omitted.

The display control function 115 displays the medical information image with the changed transparency in the display area DA of the AR glasses 151. Specifically, the display control function 115 outputs the medical information image with the changed transparency, and a control signal for displaying the medical information in the display area DA of the AR glasses 151 to the transmission function 116. The transmission function 116 outputs the medical information image with the changed transparency, and the control signal for displaying the medical information in the display area DA of the AR glasses 151 to the medical information display apparatus 152. Because the medical information processing according to the present application example is executed in the X-ray diagnostic apparatus 191 except that the medical information image with the reduced transparency is displayed on the AR glasses 151 using the control function 159, the description thereof will be omitted. In addition, because effects in the present application example are also similar to those in the embodiment and the first to seventh modified examples, the description thereof will be omitted.

In a case where the technical idea according to the present embodiment is implemented by the medical image collection system 100, the medical image collection system 100 includes the transmissive display area DA, includes the AR glasses 151 that can display medical information in the display area DA, and the medical image collection apparatus 110 that collects X-ray images related to the subject P by X-ray image capturing and/or fluoroscopy of the subject P, acquires the medical information from the medical image collection apparatus 110, changes the transparency of a medical information image indicating the medical information in accordance with an operation performed on the medical image collection apparatus 110, and displays the medical information image with the changed transparency in the display area DA. Because a processing procedure of medical information display processing in the medical image collection system 100 corresponds to that of the embodiment, the description thereof will be omitted. In addition, because the effects of the medical image collection system 100 are similar to those described in the embodiment, the first to seventh modified examples, and the application example, the description thereof will be omitted.

In a case where the technical idea according to the present embodiment is implemented by a medical information display control method, the medical information display control method includes acquiring medical information to be displayed in a transmissive display area on the AR glasses 151, changing the transparency of a medical information image indicating the medical information in accordance with an operation performed on the medical image collection apparatus 110, and displaying the medical information image with the changed transparency in the display area DA. Because a processing procedure of medical information display processing executed by the medical information display control method corresponds to that of the embodiment, the description thereof will be omitted. In addition, because the effects of the medical information display control method are similar to those described in the embodiment, the first to seventh modified examples, and the application example, the description thereof will be omitted.

In a case where the technical idea according to the embodiment is implemented by a medical information display control program, the medical information display control program causes a computer to execute acquiring medical information to be displayed in the transmissive display area DA of the AR glasses 151, changing the transparency of a medical information image indicating the medical information in accordance with an operation performed on the medical image collection apparatus 110, and displaying the medical information image with the changed transparency in the display area DA.

For example, by installing the medical information display control program to a computer of the medical information display apparatus 150, the medical image collection apparatus 191, or the like and loading the medical information display control program into a memory, it is possible to implement medical information display processing. At this time, a program capable of causing the computer to execute the medical information display processing may be stored in a storage medium such as a magnetic disk (hard disk, or the like), an optical disk (compact disc read-only memory (CD-ROM), a DVD, or the like), or a semiconductor memory and distributed. In addition, distribution of the medical information display control program is not limited to distribution via the above-described medium, and may be distributed, for example, by using a telecommunication function such as downloading via the Internet. Because procedures and effects in the medical information display processing to be implemented by the medical information display control program are similar to those in the embodiment, the first to seventh modified examples, and the application example, the description thereof will be omitted.

According to at least the embodiment, the first to seventh modified examples, the application example, and the like described above, it is possible to provide medical information in a manner appropriate for the user in accordance with a usage scene of a medical device.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information display apparatus comprising:
augmented reality glasses including a transmissive display area, the augmented reality glasses being capable of displaying medical information in the display area; and
processing circuitry configured to
acquire the medical information from a medical image collection apparatus,
change a transparency of a medical information image indicating the medical information in accordance with an operation performed on the medical image collection apparatus, and
display the medical information image with the changed transparency in the display area.

2. The medical information display apparatus according to claim 1, wherein the operation is at least one of an operation related to collection of a medical image performed by the medical image collection apparatus, and an action of a user wearing the augmented reality glasses.

3. The medical information display apparatus according to claim 1, wherein the medical information is at least one of a medical image collected by the medical image collection apparatus and/or a medical device related to the medical image collection apparatus, collection information related to collection of the medical image, and a movable range of a movable part in the medical image collection apparatus.

4. The medical information display apparatus according to claim 1,
wherein the medical image collection apparatus is an X-ray diagnostic apparatus,
wherein the operation is an execution instruction of fluoroscopy with respect to a subject,
wherein the medical information is a fluoroscopic image generated by the fluoroscopy, and
wherein the processing circuitry reduces the transparency of the fluoroscopic image in accordance with the execution instruction of the fluoroscopy and displays the fluoroscopic image with the reduced transparency in the display area.

5. The medical information display apparatus according to claim 1,
wherein the medical image collection apparatus is an X-ray diagnostic apparatus,
wherein the operation corresponds to a line of sight of a user wearing the augmented reality glasses and/or a front side of a head of the user deviating from the X-ray diagnostic apparatus during execution of fluoroscopy of a subject,
wherein the medical information is a fluoroscopic image generated by the fluoroscopy, and
wherein the processing circuitry reduces the transparency of the fluoroscopic image in accordance with the line of sight and/or the front side of the head deviating from the X-ray diagnostic apparatus during execution of the fluoroscopy, and displays the fluoroscopic image with the reduced transparency in the display area.

6. The medical information display apparatus according to claim 3,
wherein the medical image collection apparatus is an X-ray diagnostic apparatus,
wherein the operation is an instruction of image capturing of a subject,
wherein the medical information is a captured image generated by the image capturing, and
wherein the processing circuitry reduces the transparency of the captured image in accordance with the instruction of the image capturing, and displays the captured image with the reduced transparency in the display area.

7. The medical information display apparatus according to claim 3,
wherein the medical image collection apparatus is an X-ray diagnostic apparatus,
wherein the operation is a stop instruction of fluoroscopy with regard to a subject,
wherein the processing circuitry acquires the stop instruction,
wherein the medical device is an image collection device capable of collecting an image of the subject from an inside of the subject,
wherein the medical information is an internal image of the subject generated by the image collection device, and
wherein the processing circuitry reduces the transparency of the internal image of the subject in accordance with the stop instruction, and displays the internal image of the subject with the reduced transparency in the display area.

8. The medical information display apparatus according to claim 4, wherein the processing circuitry makes a brightness of an external region of the fluoroscopic image in the display area darker than a brightness of the fluoroscopic image in accordance with the execution instruction of the fluoroscopy.

9. The medical information display apparatus according to claim 3,
wherein the medical image collection apparatus is an X-ray diagnostic apparatus,
wherein the operation is an instruction of fluoroscopy or image capturing with regard to a subject,
wherein the medical information is a fluoroscopic image generated by the fluoroscopy or a captured image generated by the image capturing,
wherein the collection information is a pictogram indicating X-ray irradiation, and
wherein the processing circuitry reduces the transparency of the pictogram and the transparency of the fluoroscopic image or the captured image in accordance with the instruction of the fluoroscopy or the image capturing, and displays the pictogram with the reduced transparency in the display area together with the fluoroscopic image or the captured image with the reduced transparency.

10. The medical information display apparatus according to claim 3,
wherein the medical image collection apparatus is an X-ray diagnostic apparatus,
wherein the operation is an instruction of fluoroscopy or image capturing with regard to a subject,
wherein the medical information is a fluoroscopic image generated by the fluoroscopy or a captured image generated by the image capturing,
wherein the collection information is a vital sign of a subject related to the medical image, and
wherein the processing circuitry reduces the transparency of an image indicating the vital sign and the transparency of the fluoroscopic image or the captured image in accordance with the instruction of the fluoroscopy or the image capturing, and displays the image indicating the vital sign with the reduced transparency in the display area together with the fluoroscopic image or the captured image with the reduced transparency.

11. The medical information display apparatus according to claim 3,
wherein the medical image collection apparatus is an X-ray diagnostic apparatus,
wherein the operation is an instruction of fluoroscopy or image capturing with regard to a subject,
wherein the collection information is X-ray irradiation information related to emission of X-rays toward the subject, and
wherein the processing circuitry reduces the transparency of an image indicating the X-ray irradiation information in accordance with the instruction of the fluoroscopy or the image capturing, and displays the image indicating the X-ray irradiation information with the reduced transparency in the display area.

12. The medical information display apparatus according to claim 3,
wherein the medical image collection apparatus is an X-ray diagnostic apparatus,
wherein the operation is a movement instruction of the movable part, and
wherein the processing circuitry reduces the transparency of an image indicating the movable range in accordance with the movement instruction, and displays the image indicating the movable range with the reduced transparency on the augmented reality glasses.

13. An X-ray diagnostic apparatus comprising processing circuitry configured to:
acquire medical information to be displayed in a transmissive display area of augmented reality glasses; and
change a transparency of a medical information image indicating the medical information in accordance with an operation performed on the X-ray diagnostic apparatus, and display the medical information image with the changed transparency in the display area.

14. A medical information display control method comprising:
acquiring medical information to be displayed in a transmissive display area of augmented reality glasses; and
changing a transparency of a medical information image indicating the medical information in accordance with an operation performed on a medical image collection apparatus, and displaying the medical information image with the changed transparency in the display area.
